(19) United States
(12) Patent Application Publication
(10) Pub. No.: US 2024/0209324 A1
(43) Pub. Date: Jun. 27, 2024

(54) VIRUS SEPARATION

(71) Applicant: Cytiva US LLC, Marlborough, MA (US)

(72) Inventor: Morven MCALISTER, Port Washington, NY (US)

(21) Appl. No.: 18/087,173

(22) Filed: Dec. 22, 2022

(51) Int. Cl. C12N 7/00

(52) U.S. Cl. CPC C12N 7/00; C12N 2750/14151

(57) ABSTRACT

A method for separating smaller viruses from larger viruses from a fluid is provided, the method comprising (a) passing fluid containing smaller and larger viruses through a porous filter comprising a porous medium at a differential pressure of at least 20 psid and obtaining the smaller viruses; (b) reducing the pressure to 3 psid or less; and (c) passing additional fluid containing viruses through the porous filter comprising the porous medium at a pressure of at least 20 psid and obtaining additional smaller viruses.

### BACKGROUND OF THE INVENTION

Some processes for separating desired viruses from fluids have exhibited reduced yields and/or yields with contamination with undesirable other viruses.

There is a need for improved methods for improving the separation of desired viruses from virus-containing fluid.

The present invention provides for ameliorating at least some of the disadvantages of the prior art. These and other advantages of the present invention will be apparent from the description as set forth below.

### BRIEF SUMMARY OF THE INVENTION

An aspect of the invention provides a method for separating viruses from a fluid, the method comprising (a) passing fluid containing viruses through a porous filter comprising a porous medium at a differential pressure of at least 20 psid and obtaining viruses: (b) reducing the pressure to 3 psid or less: and (c) passing additional fluid containing viruses through the porous medium at a pressure of at least 20 psid and obtaining additional viruses.

### DETAILED DESCRIPTION OF THE INVENTION

In accordance with an aspect of the invention, a method for separating viruses from a fluid is provided, the method comprising (a) passing fluid containing viruses through a porous filter comprising a porous medium at a differential pressure of at least 20 psid and obtaining viruses: (b) reducing the pressure to 3 psid or less: and (c) passing additional fluid containing viruses through the porous filter comprising the porous medium at a pressure of at least 20 psid and obtaining additional viruses.

In one aspect, the pressure in (b) is 0 psid.

In typical aspects, in (b) the reduced pressure is for at least 2 minutes or at least 5 minutes and/or the pressure in (a) and (c) is 30 psid, or in the range of from 20 psid to 40 psid. In some aspects, in (b) the reduced pressure is for in the range of 2 minutes to 15 minutes.

In preferred aspect of the method, the separated viruses are adeno-associated viruses (AAVs), including 11 AAV serotypes.

Advantageously, smaller viruses (e.g., AAVs) can be separated (e.g., purified) with minimal or no impact on the removal of larger adventitious viruses (e.g., larger than about 50 nm, such as retroviruses) from the fluid being filtered. For example, the recovery of separated AAV (separated from larger viruses) can be increased by at least 1 log, in some aspects over 2 logs, while mitigating the risk of contamination by large viruses. With respect to large virus removal, using PR772 (about 82 nm in size) as a representative large virus, the porous medium typically achieves a log reduction value (LRV) of at least 6 logs of the large virus, e.g., calculated from titers of phage content of starting material and in the pooled effluent.

A variety of porous filters comprising porous media and filter devices including porous filters comprising porous media (including those that are commercially available, suitable filters can include more than 1 membrane/layer of porous media) can be used in accordance with aspects of the invention. The porous media can have any suitable pore structure, e.g., a pore size (for example, as evidenced by bubble point, or by K_{L} as described in, for example, U.S. Pat. No. 4,340,479, or evidenced by capillary condensation flow porometry), a pore rating, a pore diameter (e.g., when characterized using the modified OSU F2 test as described in, for example, U.S. Pat. No. 4,925,572), or removal rating that allows the passage therethrough of one or more classes/types of smaller viruses of interest while minimizing/preventing the passage therethrough of undesirable materials and/or undesirable classes/types of larger viruses as the fluid passes through the porous medium.

In a preferred aspect wherein the obtained smaller viruses have been separated from larger viruses in the fluid, the smaller viruses are smaller than 40 nanometers in size.

In a preferred aspect, the porous media (membranes) prevent the passage therethrough of viruses having a size of about 45 nanometers or larger.

Preferred filter devices including porous filters comprising porous media suitable for use in aspects of the invention include Ultipor® DV50 VF grade virus removal filters (Pall Corporation, Port Washington, NY). Other suitable filer devices include those described in, for example, U.S. Pat. Nos. 7,318,800 and 6,113,784.

The porous medium/media can have any desired critical wetting surface tension (CWST, as defined in, for example, U.S. Pat. No. 4,925,572), and as additionally disclosed in, for example, U.S. Pat. Nos. 5,152,905, 5,443,743, 5,472,621, and 6,074,869. Typically, the medium is water wettable, e.g., a CWST of 72 dynes/com (72×10⁻⁵ N/cm) or greater.

A variety of membranes, including commercially available membranes, are suitable for use as porous media in aspects of the invention, including membranes available from Pall Corporation (Port Washington, NY).

In accordance with aspects of the invention, the porous filter/porous medium can have a variety of configurations, including planar (flat), pleated, hollow cylindrical, and hollow pleated cylindrical.

In some aspects, the filter device can include, in addition to the porous filter, additional elements, layers, or components, that can have different structures and/or functions, e.g., at least one of any one or more of the following: prefiltration, support, drainage, spacing and cushioning. Illustratively, the filter device, or filter including the porous medium, can also include at least one additional element such as a mesh and/or a screen.

The porous filter/porous medium is typically disposed in a housing comprising at least one inlet and at least one outlet and defining at least one fluid flow path between the inlet and the outlet, wherein the porous medium or filter including the porous medium is across the fluid flow path, to provide a filter device. Preferably, the filter device is sterilizable. Any housing of suitable shape and providing at least one inlet and at least one outlet may be employed.

The housing can be fabricated from any suitable rigid impervious material, including any impervious thermoplastic material, which is compatible with the fluid being processed. Typically, the housing is fabricated from a polymer. In an aspect, the housing is a polymer, in some aspects, a transparent or translucent polymer, such as an acrylic, polypropylene, polystyrene, or a polycarbonated resin. Such a housing is easily and economically fabricated, and allows observation of the passage of the fluid through the housing.

The following example further illustrates the invention but, of course, should not be construed as in any way limiting its scope.

### EXAMPLE

This example demonstrates increased separation (and recovery) of smaller viruses while minimizing the presence of larger viruses in the filtered fluid according to an aspect of the invention, wherein bacteriophages are utilized as surrogates for AAVs.

Ultipor® DV50 filter devices (Pall Corporation, Port Washington, NY) comprising porous media are obtained.

Phosphate buffered saline (PBS) containing 0.02% v/v Tween 80 is inoculated with both 10⁷ PFU/mL PR772 (about 82 nm in size) and 10⁷ PFU/mL PP7 (about 25 nm in size).

The inoculated fluid is mixed, and a sample is taken to determine pre-filtration levels. The fluid is split into two separate volumes to allow testing at two different test pressures (20 psi and 30 psi). Testing is carried out as set out in the following table:

| | Total | Test Pressure/Depressurization Duration | | | |
|---|---|---|---|---|---|
| | Accumulated | 30 psi, | 30 psi, | 20 psi, | 20 psi, |
| | Vol. (L/m²) | 2 min | 10 min | 2 min | 10 min |
| PP7 | | 3.80E+07 | 3.80E+07 | 3.80E+07 | 3.80E+07 |
| Concen- | 22.5 | | | | |
| tration | 113 | 3.30E+06 | 2.60E+06 | 8.20E+06 | 7.80E+06 |
| (PFU/mL) | Depres- | 2 | 10 | 2 | 5 |
| | surization | | | | |
| | Duration | | | | |
| | (min) | | | | |
| | 122* | 1.10E+08 | 1.30E+08 | 9.00E+07 | 1.60E+08 |
| | 131 | | | | |
| PR772 | | 6.40E+07 | 6.40E+07 | 5.60E+07 | 5.40E+07 |
| Concen- | 22.5 | | | | |
| tration | 113 | 0.00E+00 | 0.00E+00 | 0.00E+00 | 0.00E+00 |
| (PFU/mL) | Depres- | 2 | 10 | 2 | 5 |
| | surization | | | | |
| | Duration | | | | |
| | (min) | | | | |
| | 122* | 0.00E+00 | 0.00E+00 | 0.00E+00 | 0.00E+00 |
| | 131 | | | | |

*Post-depressurization

A post-filtration input sample is also removed to account for any losses in bacteriophage titers during testing. All aliquots are assayed for PR772 and PP7.

The larger bacteriophage (PR772, approximately 82 nm) is 100% retained at both initial pressures and after the pressure reduction and subsequent pressures.

After depressurization and subsequent repressurization, an increase in PP7 yield of about 1.7 logs is observed at 20 psid and 30 psid.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and “at least one” and similar referents in the context of describing the invention (especially in the context of the following claims) are to be construed to cover both the singular and the plural, unless otherwise indicated herein or clearly contradicted by context. The use of the term “at least one” followed by a list of one or more items (for example, “at least one of A and B”) is to be construed to mean one item selected from the listed items (A or B) or any combination of two or more of the listed items (A and B), unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred aspects of this invention are described herein, including the best mode known to the inventors for carrying out the invention. Variations of those preferred aspects may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover. any combination of the above-described elements in all possible variations thereof is encompassed by the invention unless otherwise indicated herein or otherwise clearly contradicted by context.

1. A method for separating viruses from a fluid, the method comprising
(a) passing fluid containing viruses through a porous filter comprising a porous medium at a differential pressure of at least 20 psid and obtaining viruses;
(b) reducing the pressure to 3 psid or less; and,
(c) passing additional fluid containing viruses through the porous filter comprising a medium at a pressure of at least 20 psid and obtaining additional viruses.

2. The method of claim 1 comprising separating larger viruses from smaller viruses from the fluid, wherein the obtained additional viruses are the smaller viruses.

3. The method of claim 1, wherein the pressure in (b) is 0 psid.

4. The method of claim 1, wherein in (b) the reduced pressure is for at least 2 minutes.

5. The method of claim 1, wherein in (b) the reduced pressure is for at least 5 minutes.

6. The method of claim 1, wherein in (b) the reduced pressure is in the range of 2 minutes to 15 minutes.

7. The method of claim 1, wherein the pressure in (a) and (c) is in at least 30 psid.

8. The method of claim 1, wherein the pressure in (a) and (c) is in the range of from 20 psid to 40 psid.

9. The method of claim 1, wherein the obtained additional viruses are AAV.

10. The method of claim 2, wherein the pressure in (b) is 0 psid.

11. The method of claim 2, wherein in (b) the reduced pressure is for at least 2 minutes.

12. The method of claim 2, wherein in (b) the reduced pressure is for at least 5 minutes.

13. The method of claim 2, wherein in (b) the reduced pressure is in the range of 2 minutes to 15 minutes.

14. The method of claim 2, wherein the pressure in (a) and (c) is in at least 30 psid.

15. The method of claim 2, wherein the pressure in (a) and (c) is in the range of from 20 psid to 40 psid.

16. The method of claim 2, wherein the obtained additional viruses are AAV.
